(19) United States
(12) Patent Application Publication
(10) Pub. No.: US 2008/0208171 A1
(43) Pub. Date: Aug. 28, 2008

(54) DEVICE AND METHOD FOR REMOVING EDEMA

(76) Inventors: Louis C. Argenta, Winston-Salem, NC (US); Michael J. Morykwas, Winston-Salem, NC (US); Lawrence X. Webb, Winston-Salem, NC (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 11/678,403

(22) Filed: Feb. 23, 2007

(51) Int. Cl. A61M 1/00

(52) U.S. Cl. 604/540

(57) ABSTRACT

The present invention relates to a device and method for removing edema from an edematous tissue. The device includes at least one aciculated component disposed in fluid communication with a suction manifold. A suction source is fluidically connected to the suction manifold to provide a suction force to the suction manifold. The suction force is transmitted through the suction manifold to the at least one aciculated component to withdraw the edema from edematous tissue through the suction manifold and the at least one aciculated component.

### FIELD OF THE INVENTION

The present invention relates generally to a device and method for removing edema, and more particularly, but not exclusively, to a device and method for removing edema using aciculated components embedded in a porous material to withdraw edema from a tissue through the aciculated components and into the porous material.

### BACKGROUND OF THE INVENTION

A common problem affecting patients is the build up of interstitial fluid between soft tissue cells which can cause swelling and irritation, prevent proper blood flow, and cause significant discomfort. This buildup of interstitial fluid, or edema, can be present in a variety of organ tissues. The medical community has recognized that immobility, sodium, medication, renal and heart problems can aggravate edema, with immobility representing a principal aggravating factor. While a patient's disease or injury may itself cause edema, prolonged immobilization can further instigate the formation of edema.

In addition to immobilization, consumption of excess sodium also upsets the natural balance of interstitial fluid. While consuming salt is usually not sufficient to cause edema by itself, excess sodium can aggravate other edemic conditions. Moreover, in addition to diet, exercise, and medication, edema is often caused by a variety of medical conditions. Problems with the heart can lead to the increase of interstitial fluid. Kidney problems reduce the body's ability to regulate sodium and water. Low protein levels caused by thyroid or liver disease also can lead to edema. Edema can also be caused by problems with the lymphatic system, because the lymphatic system is generally responsible for maintenance of proper interstitial fluid balance. Thus, edema can be caused by a wide variety of factors, and as such, is a prevalent problem among segments of the patient population.

While edema is simply the presence of excess fluid outside of the cells, treating edema has proven to be very difficult. Current treatment regimens include simple steps like reducing sodium intake, losing weight, exercising regularly, avoiding prolonged travel without breaks, the use of diuretics to lower interstitial fluid volume. Current treatments such as these have been helpful in reducing edema for suffering patients, but many of these solutions take a long period of time to produce results and require patients to make lifestyle changes. In many cases, a patient suffering from edema is not capable of exercising regularly, or is not willing to reduce sodium consumption. Patients may not wish to take medications regularly. Thus, many patients still suffer from edema, because they are unwilling or unable to follow indicated treatments. This is particularly true if the patient is suffering from a disease or injury.

While edema can be caused by many factors, edema is a particular problem for patients who have large wounds. Treating large wounds in the skin or subcutaneous tissue presents a physician with a variety of complications. When a patient's skin is badly injured the contents of the skin cells spill out into the surrounding tissue, increasing interstitial fluid. Edema at a wound retards the proper inflow of blood and nutrients to the wound. Without proper blood flow, the tissue becomes ischemic and may die. Dead tissue in combination with a dearth of white blood cells greatly increases the risk for bacterial infection. Infection can cause additional cell death, leading to more cell lysis, and more edema, making healing the wound even more difficult.

For reasons such as these, there remains a need in the medical field to reduce and remove edema.

### SUMMARY OF THE INVENTION

The present invention relates to a device and method for removing edema from tissues such as subcutaneous tissue, submuscular tissue, and/or intramuscular space, and may be used on intact or broken tissue, e.g., a closed or an open wound. In one exemplary configuration of the present invention, a device for removing edema from an edematous tissue includes at least one aciculated (i.e., needlelike) component structured to pierce an edematous tissue at a treatment location. The aciculated component may comprise a pointed, tubular component, such as a needle or catheter. The aciculated component is structured to fluidically communicate with the edematous tissue to permit the passage of edema through the aciculated component. The device also includes a suction manifold having a treatment surface for placement in facing opposition to the treatment location. The suction manifold is disposed in fluid communication with the at least one aciculated component, so that the suction manifold can receive edema transported through the aciculated component from the edematous tissue. As part of the device, a suction source is provided in fluid communication with the suction manifold to provide a suction force to the suction manifold. The suction force is transmitted through the suction manifold to the at least one aciculated component to withdraw the edema from edematous tissue through the suction manifold and the at least one aciculated component.

The device of the present invention may desirably include a cover disposed over the suction manifold. The cover may be made of a rigid, semi-rigid or flexible material and may serve a variety of functions such as protecting the area from contaminants, stabilizing the aciculated components and suction manifold, and creating an air tight seal. In one particular configuration, the cover may be provided in the form of a flexible self-adhesive sheet. The edema removal device may also include a conduit or tube connecting the suction source to the suction manifold.

In another aspect of the present invention, a method is provided for removing edema. The method includes providing an appliance comprising at least one aciculated component and a suction manifold. The aciculated component is structured to pierce an edematous tissue at a treatment location and structured to fluidically communicate with the edematous tissue to permit the passage of edema through the aciculated component. The suction manifold has a treatment surface for placement in facing opposition to the treatment location and is disposed in fluid communication with the at least one aciculated component to receive edema transported through the aciculated component from the edematous tissue. The method also includes placing the appliance at the treatment location and piercing the edematous tissue with the at least one aciculated component. As part of the method, a suction source is operably connected in fluid communication with the suction manifold to provide a suction force to the suction manifold and the at least one aciculated component, and edema is withdrawn from the edematous tissue through the suction manifold and the at least one aciculated component.

In a further aspect of the present invention, a method for removing edema from an edematous tissue is provided comprising the steps of inserting at least one aciculated component into an edematous tissue, attaching the at least one aciculated component in fluid communication with a suction manifold, and applying a suction force through the suction manifold to the at least one aciculated component to draw edema from the edematous tissue through the at least one aciculated component and the suction manifold.

### BRIEF DESCRIPTION OF THE DRAWINGS

FIG. 1 is a schematic side elevational view taken in partial cross-section showing an exemplary configuration of the present invention disposed above an edematous tissue.

FIG. 2 is a schematic side elevational view in partial cross-section showing an exemplary configuration of the present invention similar to that of FIG. 1, but having a pass-through port in the cover.

FIG. 3 is a schematic side elevational view in partial cross-section showing an exemplary configuration of the present invention disposed within an open wound.

FIG. 4 is a schematic side elevational view in partial cross-section showing an exemplary device of the present invention having a bifurcated conduit.

FIG. 4A is a schematic perspective view of a fenestrated aciculated component of the present invention.

### DETAILED DESCRIPTION OF THE INVENTION

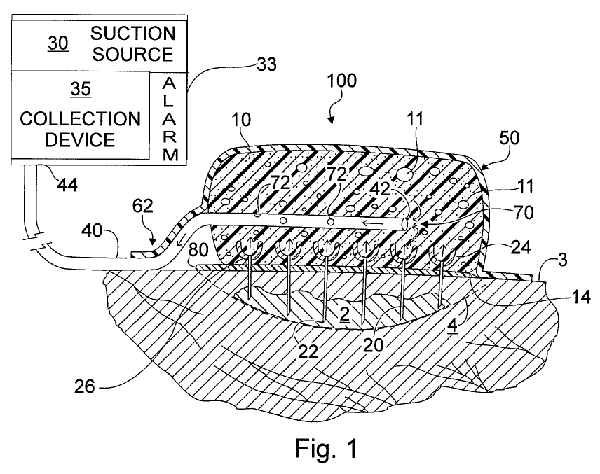

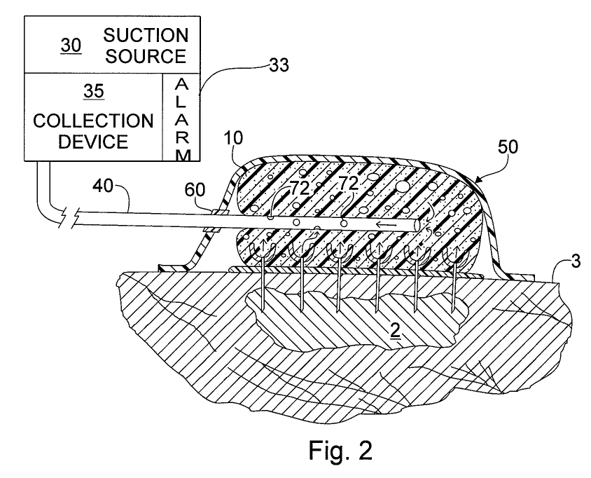

Referring now to the figures, wherein like elements are numbered alike throughout, an exemplary device for removing edema, generally designated 100, is provided, as shown, for example, in FIG. 1. The edema removal device 100 includes one or more aciculated components 20, such as needles, for withdrawing edema from edematous tissue 2. The aciculated components 20 have a proximal end 24 embedded within a suction manifold 1 0, which may comprise a porous material, such as an open-cell foam. The suction manifold 10 and the aciculated components 20 are in fluid communication so that fluid, e.g., edema, can flow from the aciculated components 20 into the suction manifold 10. A suction source 30 is fluidically connected to the suction manifold 10 via a conduit 40 having the distal end 42 in fluid communication with the suction manifold 10 and a proximal end 44 in fluid communication with the suction source 30. Upon activation of the suction source 30, a suction force is transmitted through the conduit 40 and through the suction manifold 10 to the aciculated components 20. The transmitted suction force causes edema to be withdrawn from the edematous tissue 2 into the aciculated components 20, through the suction manifold 10 and conduit 40, and into a collection device 35 of the suction source 30. Optionally, the edema removal device 100 includes a cover 50 placed over the suction manifold 10 and aciculated components 20 and sealed into position about the edematous tissue 2 to contain the suction force thereunder.

Turning to the figures in more detail, FIG. 1 schematically illustrates a side elevational view in partial cross-section of an edema removal device 100 in accordance with the present invention. The edema removal device 100 includes at least one aciculated component 20 suitable for insertion into an edematous tissue 2 and for removing edema from the edematous tissue 2. In this regard, the aciculated components 20 may be dimensioned and configured with regard to the location and nature of the edema to be removed. For example, the length of the aciculated components 20 may be selected with regard to the depth into the tissue 2 that the edema extends. In particular, for certain pathologies the distribution of the edema in the edematous tissue 2 may present in a known configuration, in which case the arrangement of the aciculated components 20 may be matched to the distribution of the edema. For instance, the edema may have a known depth profile 4, extending deeper at the center the edematous region than at the periphery. In such a case, longer aciculated components 20 may be provided at the center of the edema removal device 100 and shorter ones at the edge. Likewise, the lateral distribution of aciculated components 20 across the surface of the device 100 may be varied to provide a closer packing density of aciculated components 20 in regions in which a greater amount of edema is expected to be encountered.

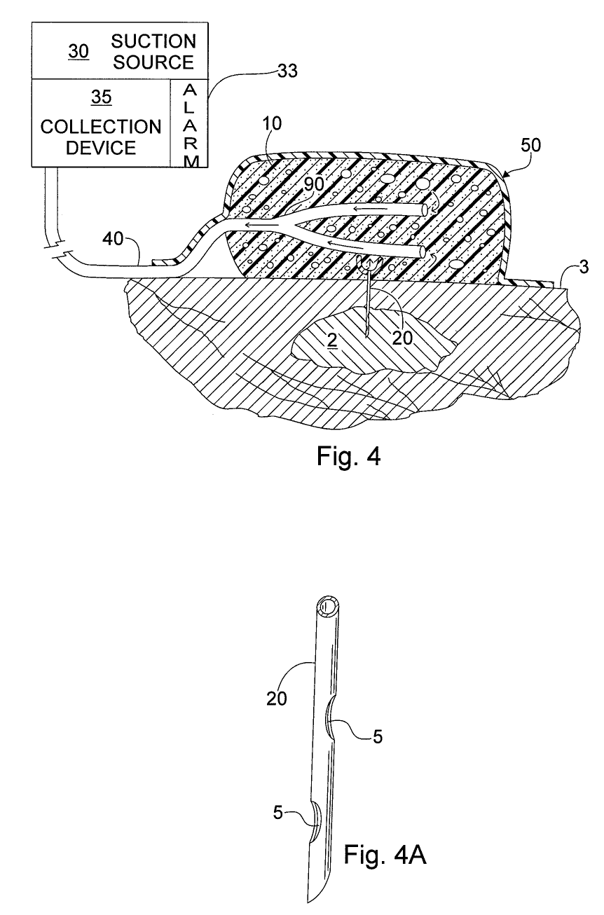

The aciculated components 20 may be provided in the form of a needle, wick, catheter, angiocath, or combinations thereof, as well as other structures suitable for removing edema from edematous tissue 2. The diameter of the aciculated components 20 may be selected with regard to the type of tissue into which the aciculated components 20 are to be inserted, as well as with regard to the volume and viscosity of the edema to be removed. In addition, the aciculated components 20 may be fenestrated with holes 5 along at least a portion of their length that is in contact with the edematous tissue 2 to assist in the removal of the edema along the fenestrated length of the aciculated components 20, as shown in FIG. 4A.

The removal of edema from the edematous tissue 2 via the aciculated components 20 is effected by application of a suction force to the aciculated components 20. The suction force is delivered to the aciculated components 20 by a suction manifold 10 disposed in fluid communication with the aciculated components 20, which directs the flow of edema from the edematous tissue 2 into the suction manifold 10. The suction manifold 10 in turn is connected in fluid communication with a suction source 30 which applies a suction force to the suction manifold 10. The aciculated components 20 are directly or indirectly connected to the suction manifold 10 at a treatment surface 14 of the manifold 10 by any method that permits fluid communication between the aciculated components 20 and the suction manifold 10. For example, the aciculated components 20 may have a proximal end 24 embedded or punched into the suction manifold 10. Alternatively, the aciculated components 20 may be attached indirectly via a connector, or screwed or snapped into the suction manifold 10, and so forth, so long as fluid communication is provided between the aciculated components 20 and the suction manifold 10. As shown in FIG. 1, the aciculated components may, for example, include a connector or anchor 26 in the form of Luer fittings for inserting into the manifold and connecting and/or for anchoring the aciculated components thereto.

The suction manifold 10 includes one or more passageways to transmit the suction force from the suction source 30 to the aciculated components 20. For example, the suction manifold 10 may desirably include a porous material such as a sponge or open-cell foam, e.g. a polyurethane foam or polyvinyl alcohol foam (PVA), having a series of interconnected pores 11 that form passageways for delivering a suction force to the aciculated components 20. A suction manifold 10 may comprise any material suitable for delivering a suction force and for permitting the flow of edema therethrough. For example, the suction manifold 10 may comprise a polymer material, such as a synthetic polymer material. In addition, the suction manifold 10 may be composed of a material that will dissolve over a period of time, and/or may be composed of an antimicrobial or antibiotic materials. The manifold may desirably include a hydrophobic material to facilitate liquid transfer through the manifold or through the connecting poses or open cells of the manifold. Exemplary materials that may be used for the suction manifold 10, alone or in combination with other materials, include V.A.C.® GranuFoam® and V.A.C.® WhiteFoam™ dressings (Kinetic Concepts Inc. San Antonio, Tex.), and the like.

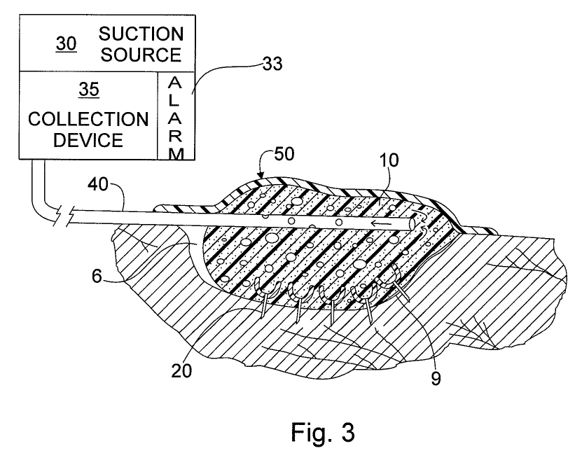

In addition, the suction manifold 10 may include a material that allows for gas and/or liquid exchange between the suction manifold 10 and the tissue, such as skin 3, adjacent the suction manifold 10. In this regard, the suction manifold 10 may be perforated or punched to enhance gas exchange or to reduce the weight of the suction manifold 10. Furthermore, the material of the suction manifold 10 may desirably permit absorption of liquids such as edema, exudate, and perspiration that may contact the suction manifold 10. Such a configuration may be desirable in cases in which the suction manifold 10 is disposed in an open wound 9, as illustrated in FIG. 3, and may permit delivery of antibiotics or antimicrobial compounds to the adjacent tissue.

The suction source 30 may be removably connected to the suction manifold 10 by an evacuation conduit 40, with the proximal and distal ends 44, 42 of the conduit 40 permanently or removably attached to or connected with the suction source 30 and suction manifold 10, respectively. The evacuation conduit 40 is provided in a form that promotes a fluid tight and gas tight connection between the suction source 30 and the suction manifold 10, such as a flexible tube, e.g., IV tubing. The distal end 42 of the conduit 40 may be embedded within the suction manifold 10, so that the conduit 40 is in fluid communication with the passageways or the pores 1 1 of the manifold 10. In addition, the conduit 40 may be fenestrated at the distal end embedded or positioned within the manifold to provide side openings 72 for delivering the suction force to the suction manifold 10 along the fenestrated length of the conduit 40. Further, the conduit 40 may comprise multiple branches disposed throughout the suction manifold 10 to distribute the suction force through the suction manifold 10. For example, as illustrated in FIG. 3, the conduit may be provided in the form of a bifurcated conduit 90, although additional branches could be utilized.

The suction source 30 may include a vacuum pump or other source of suction that can safely and reliably provide a suction force to the suction manifold 10 and aciculated components 20. The suction source 30 may be configured to provide a reduced pressure from 10 mm Hg below atmospheric pressure to 300 mm Hg below atmospheric pressure, for example. The suction source 30 may optionally include a collection device 35 to collect edema and other fluid from the suction manifold 10. Alternatively, a fluid collection device may be provided along the length of the conduit 40 between the suction source 30 and the suction manifold 10. The collection device may be provided in the form of a fluid trap and may include a shut-off for halting the application of the suction force to the suction manifold 10 when the collection device becomes filled with a predetermined amount of fluid. An alarm 33 may be provided to announce or indicate that the collection device is filled to a predetermined or an adjustable level or to indicate or announce a selected malfunction such as loss of suction or clogging of conduit 40. The suction source 30 may desirably include a controller for controlling the suction source 30 and optionally the alarm 33 and for providing intermittent suction in the form of alternating periods of application and non-application of suction.

Upon application of the suction force to the suction manifold 10, the suction manifold 10 may contract and rub against the tissue in contact with the suction manifold 10. Movement between the suction manifold 10 and the tissue contacted by the suction manifold 10 may irritate the contacted tissue, especially during application of intermittent suction. To alleviate potential irritation, the edema removal device 100 of the present invention may optionally include a friction-reducing layer 80 disposed between the suction manifold 10 and the adjacent tissue, such as skin 3, to reduce friction between the suction manifold 10 and the adjacent tissue. The friction-reducing layer 80 may be porous, e.g. a synthetic polymer mesh, or non-porous and may also provide additional protection of the wound from microbes and debris. Various material having a low coefficient of friction can be used to construct the friction-reducing layer 80. For example, the friction-reducing layer 80 may include a non-adherent material, such as an ointment-impregnated gauze or a synthetic or natural polymer material. Exemplary materials suitable for use as the friction-reducing layer 80 include Aquaphor® Gauze (Smith & Nephew, Largo, Fla.), Adaptic® Non-Adherent Dressing (New Brunswick, N.J.), and so forth. The friction-reducing layer 80 may be provided as a separate component disposed between the suction manifold 10 and the adjacent tissue. Alternatively, a friction-reducing coating may be disposed on the lower surface 14 of the suction manifold 10 for contact with adjacent tissue.

To assist in the application of the suction force by the suction manifold 10, the edema removal device 100 may desirably include a cover 50 disposed over the suction manifold 10 and sealed in location to tissue, such as skin 3, proximate the edematous tissue 2 so that the cover 50 promotes containment of the suction force thereunder. The contained suction force under the cover 50 may desirably create a negative pressure (or sub-atmospheric) environment about the treatment location to provide negative pressure therapy at the treatment location. For example, if the treatment location is an open wound 9, the introduction of a suction force under the sealed cover 50 may provide negative pressure wound therapy at the wound 9. The cover 50 includes a port through which the conduit 40 communicates with the space under the cover 50. The port may comprise an edge 62 of the cover 50 under which the conduit 40 passes, such as a mesentery, for example. Alternatively, the port may be provided as an opening 60, such as a nipple or plug, disposed at the surface of the cover 50, or other suitable structure.

The use of a cover 50 may be particularly desirable in the case where the suction manifold 10 comprises a porous open-cell material having passageways which communicate with exterior surfaces of the suction manifold 10. In such a case, the cover 50 may seal the ends of passageways that terminate at the upper and side surfaces of the suction manifold 10, leaving the passageways at the outer periphery open only at the lower treatment surface 14 of the suction manifold 10 facing the edematous tissue 2. The inclusion of a cover 50 may also help prevent debris from entering the treatment site and may provide a compression force against the suction manifold 10 and the treatment site. If the edema removal device 100 is used over an open wound 9, the cover 50 may also provide protection against infection. The selection of an appropriate cover material will be influenced by factors such as durability, the ability to protect the wound, gas permeability, and the ability to maintain the relative position of the suction manifold 10 to the edematous tissue 2.

The cover 50 may be conveniently provided in the form of a self-adhesive cover 50, such as a flexible polymer sheet or surgical drape, for example. Exemplary self-adhesive drapes include Ioban® drape (3M Corporation, St. Paul, Minn.), OpSite® drape (Smith & Nephew, Largo, Fla.), and so forth. Providing the cover 50 in the form of a flexible sheet or surgical drape may be particularly desirable for use in applications where the cover 50 should conform to the treatment site. Further, the use of a flexible sheet or drape may be particularly desirable in weight dependent locations. Providing a self-adhesive cover 50 may simplify the application of the cover 50 to the tissue surrounding the edematous tissue 2 and may conveniently permit the cover 50 to be attached to and/or immobilize other components of the edema removal device 100 that the cover adhesive contacts, such as suction conduit 40 or suction manifold 10. Instead, if a non-adhesive cover 50 is used, a separately applied adhesive or other suitable material may be used to secure and/or seal the cover 50 to the treatment site. The adhesive may be disposed along an entire surface of the cover 50 or may be disposed only about the periphery of the cover 50. In addition to adhesives, the cover 50 may also be secured by other methods such as bands, straps, ties, casts, and so forth.

In an exemplary use, the optional friction-reducing layer 80 is placed at the treatment location against the skin 3 or the wound bed. Then, the suction manifold 10, with aciculated components 20 and conduit 40 attached is placed against the friction-reducing layer 80 and pressed so that the aciculated components 20 pierce the friction-reducing layer 80 and the tissue to be treated. Alternatively, the friction-reducing layer 80 may be provided on the surface of the manifold. Furthermore, the conduit 40 may be attached to the suction manifold 10 either before or after the aciculated components 20 are inserted in the edematous tissue 2. A self-adhesive cover 50 is applied over the suction manifold 10, the skin 3, and the conduit 40. The adhesive secures the cover 50 to the suction manifold 10, the skin 3, and conduit 40 to form a seal at the treatment location. The conduit 40 is attached to the suction source 30, which may then be activated to withdraw edema from the edematous tissue 2. Suction is applied at a desired suction pressure and is maintained until the desired amount of edema is removed.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as set forth in the claims.

What is claimed is:

1. A device for removing edema from an edematous tissue, comprising:
a. at least one aciculated component structured to pierce an edematous tissue at a treatment location and structured to fluidically communicate with the edematous tissue to permit the passage of edema through the aciculated component;
b. a suction manifold having a treatment surface for placement at the treatment location, the suction manifold disposed in fluid communication with the at least one aciculated component to receive edema transported through the aciculated component from the edematous tissue; and
c. a suction source in fluid communication with the suction manifold to provide a suction force to the suction manifold and the at least one aciculated component to withdraw the edema from edematous tissue through the suction manifold and the at least one aciculated component.

2. The device of claim 1, wherein the at least one aciculated component comprises a plurality of aciculated components.

3. The device of claim 2, wherein at least two of the aciculated components differ in length.

4. The device of claim 2, wherein the aciculated components have substantially the same length.

5. The device of claim 2, wherein the aciculated components are positioned at the treatment surface of the suction manifold.

6. The device of claim 1, comprising a connector disposed between the suction manifold and the at least one aciculated component.

7. The device of claim 1, wherein the at least one aciculated component is directly connected to the suction manifold.

8. The device of claim 1, wherein the at least one aciculated component is fenestrated.

9. The device of claim 1, wherein the at least one aciculated component comprises a first end structured to pierce the tissue to be treated.

10. The device of claim 9, wherein the at least one aciculated component comprises a second end disposed within the suction manifold.

11. The device of claim 1, wherein the at least one aciculated component comprises a needle.

12. The device of claim 1, wherein the at least one aciculated component comprises a catheter.

13. The device of claim 1, wherein the at least one aciculated component comprises a wick.

14. The device of claim 1, wherein the at least one aciculated component comprises an angiocath.

15. The device of claim 1, wherein the suction manifold comprises a porous material.

16. The device of claim 1, wherein the suction manifold comprises an open-cell foam.

17. The device of claim 16, wherein the suction manifold comprises a polymer material.

18. The device of claim 1, wherein the suction manifold comprises a polymer material.

19. The device of claim 18, wherein the suction manifold comprises a synthetic polymer material.

20. The device of claim 1, wherein the suction manifold comprises a sponge.

21. The device of claim 1, comprising a friction-reducing layer for positioning under the suction manifold and in contact with tissue at the treatment location to reduce friction between the suction manifold and the treatment location.

22. The device of claim 21, wherein the friction-reducing layer comprises a non-adherent material.

23. The device of claim 22, wherein the non-adherent material comprises an ointment-impregnated gauze.

24. The device of claim 22, wherein the non-adherent material comprises a synthetic polymer material.

25. The device of claim 22, wherein the non-adherent layer comprises a synthetic polymer mesh.

26. The device of claim 1, comprising:
a. a cover disposed over the suction manifold;
b. a seal structured to seal and secure the cover at the treatment location;
c. an evacuation conduit for connection to the suction source, the evacuation conduit cooperating with the cover and the suction source to deliver the suction force to the suction manifold.

27. The device of claim 26, comprising a port located at the cover, and wherein the evacuation conduit fluidically communicates with the port.

28. The device of claim 26, wherein the cover comprises a flexible sheet.

29. The device of claim 28, wherein the seal includes an adhesive on a sheet surface facing the treatment location to attach and seal the sheet to tissue surrounding the treatment location.

30. The device of claim 26, wherein the conduit comprises multiple branches.

31. The device of claim 26, wherein the conduit comprises fenestrations.

32. The device of claim 1, comprising a self-adhesive cover structured to cover the suction manifold and the treatment location and structured to form a seal about the treatment location.

33. The device of claim 1, comprising a controller for operating the suction source in alternating periods of application and non-application of suction.

34. The device of claim 1, comprising a controller for controlling the operation of the suction source.

35. The device of claim 1, comprising an alarm for indicating when a predetermined amount of fluid has been removed through the suction manifold.

36. The device of claim 35, comprising a collection device for receiving edema removed through the suction manifold.

37. The device of claim 36, wherein the alarm is structure to indicate when a predetermined amount of liquid is collected in the collection device.

38. The device of claim 1, wherein the suction source is configured to provide a reduced pressure from 10 mm Hg below atmospheric pressure to 300 mm Hg below atmospheric pressure

39. A method for removing edema from an edematous tissue, comprising the steps of:
a. providing at least one aciculated component structured to pierce an edematous tissue at a treatment location and structured to fluidically communicate with the edematous tissue to permit the passage of edema through the aciculated component;
b. providing a suction manifold having a treatment surface for placement in facing opposition to the treatment location, the suction manifold disposed in fluid communication with the at least one aciculated component to receive edema transported through the aciculated component from the edematous tissue;
c. placing the appliance at the treatment location and piercing the edematous tissue with the at least one aciculated component;
d. operably connecting a suction source in fluid communication with the suction manifold to provide a suction force to the suction manifold and the at least one aciculated component; and
e. withdrawing the edema from edematous tissue through the suction manifold and the at least one aciculated component.

40. The method of claim 39, wherein the step of piercing the edematous tissue, comprise inserting at least two aciculated components at differing depths into the edematous tissue.

41. The method of claim 39, wherein the at least one aciculated component comprises a needle.

42. The method of claim 39, wherein the at least one aciculated component comprises a catheter.

43. The method of claim 39, wherein the at least one aciculated component comprises a wick.

44. The method of claim 39, wherein the at least one aciculated component comprises an angiocath.

45. The method of claim 39, wherein the suction manifold comprises a porous material.

46. The method of claim 39, wherein the suction manifold comprises an open-cell foam.

47. The method of claim 46, wherein the suction manifold comprises a polymer material.

48. The method of claim 39, wherein the suction manifold comprises a polymer material.

49. The method of claim 48, wherein the suction manifold comprises a synthetic polymer material.

50. The method of claim 39, wherein the suction manifold comprises a sponge.

51. The method of claim 39, comprising the step of placing a friction-reducing layer between the edematous tissue and the suction manifold to reduce friction between the suction manifold and the treatment location.

52. The method of claim 51, wherein the friction-reducing layer comprises a non-adherent material.

53. The method of claim 52, wherein the non-adherent material comprises an ointment-impregnated gauze.

54. The method of claim 52, wherein the non-adherent material comprises a synthetic polymer material.

55. The method of claim 52, wherein the non-adherent layer comprises a synthetic polymer mesh.

56. The method of claim 39, comprising the steps of positioning a cover over the suction manifold and sealing the cover to tissue surrounding the edematous tissue.

57. The method of claim 56, wherein the cover comprises a flexible sheet.

58. The method of claim 56, wherein the cover comprises a self-adhesive sheet structured to cover the suction manifold and the treatment location and structured to form a seal about the treatment location.

59. A method for removing edema from an edematous tissue, comprising the steps of:
a. inserting at least one aciculated component into an edematous tissue;
b. attaching the at least one aciculated component in fluid communication with a suction manifold; and
c. applying a suction force through the suction manifold to the at least one aciculated component to draw edema from the edematous tissue through the at least one aciculated component and the suction manifold.

60. The method of claim 59, wherein the at least one aciculated component comprises a needle.

61. The method of claim 59, wherein the step of inserting the at least one aciculated component, comprises inserting at least two aciculated components at differing depths into the edematous tissue.

62. The method of claim 59, wherein the at least one aciculated component comprises a catheter.

63. The method of claim 59, wherein the at least one aciculated component comprises a wick.

64. The method of claim 59, wherein the at least one aciculated component comprises an angiocath.

65. The method of claim 59, wherein the suction manifold comprises a porous material.

66. The method of claim 59, wherein the suction manifold comprises an open-cell foam.

67. The method of claim 66, wherein the suction manifold comprises a polymer material.

68. The method of claim 59, wherein the suction manifold comprises a polymer material.

69. The method of claim 68, wherein the suction manifold comprises a synthetic polymer material.

70. The method of claim 59, wherein the suction manifold comprises a sponge.

71. The method of claim 59, wherein the inserting step comprises inserting multiple aciculated components through the tissue.

72. The method of claim 59, comprising the step of placing a friction-reducing layer between the edematous tissue and the suction manifold to reduce friction between the suction manifold and the treatment location.

73. The method of claim 72, wherein the friction-reducing layer comprises a non-adherent material.

74. The method of claim 73, wherein the non-adherent material comprises an ointment-impregnated gauze.

75. The method of claim 73, wherein the non-adherent material comprises a synthetic polymer material.

76. The method of claim 73, wherein the non-adherent layer comprises a synthetic polymer mesh.

77. The method of claim 59, comprising the steps of positioning a cover over the suction manifold, and sealing the cover to tissue surrounding the edematous tissue.

78. The method of claim 77, wherein the cover comprises a flexible sheet.

79. The method of claim 77, wherein the cover comprises a self-adhesive sheet structured to cover the suction manifold and the treatment location and structured to form a seal about the treatment location.

80. A device for removing edema from an edematous tissue, comprising:
a. at least one aciculated component structured to pierce an edematous tissue at a treatment location and structured to fluidically communicate with the edematous tissue to permit the passage of edema through the aciculated component;
b. an open-cell polymer foam disposed in fluid communication with the at least one aciculated component to receive edema transported through the aciculated component from the edematous tissue; and
c. a cover disposed over the open-cell polymer foam and structured for sealing to tissue surrounding the treatment location to permit sub-atmospheric pressure to be maintained under the cover.

81. The device of claim 80, wherein the at least one aciculated component comprises a plurality of aciculated components.

82. The device of claim 80, comprising a connector disposed between the open-cell polymer foam and the at least one aciculated component.

83. The device of claim 80, wherein the at least one aciculated component is directly connected to the open-cell polymer foam.

84. The device of claim 83, wherein the at least one aciculated component comprises an end disposed within the open-cell polymer foam.

85. The device of claim 80, wherein the at least one aciculated component comprises a needle.

86. The device of claim 80, wherein the at least one aciculated component comprises a catheter.

87. The device of claim 80, wherein the at least one aciculated component comprises a wick.

88. The device of claim 80, wherein the at least one aciculated component comprises an angiocath.

89. The device of claim 80, wherein the open-cell polymer foam comprises a synthetic polymer material.

90. The device of claim 80, wherein the open-cell polymer foam comprises a sponge.

91. The device of claim 80, comprising a friction-reducing layer for positioning under the open-cell polymer foam and in contact with tissue at the treatment location to reduce friction between the open-cell polymer foam and the treatment location.

92. The device of claim 91, wherein the friction-reducing layer comprises a non-adherent material.

93. The device of claim 92, wherein the non-adherent material comprises an ointment-impregnated gauze.

94. The device of claim 92, wherein the non-adherent material comprises a synthetic polymer material.

95. The device of claim 92, wherein the non-adherent layer comprises a synthetic polymer mesh.

96. The device of claim 80, wherein the cover comprises a flexible sheet.

97. The device of claim 80, wherein the cover comprises a self-adhesive sheet structured to cover the open-cell polymer foam and the treatment location and structured to form a seal about the treatment location.
